(19) United States
(12) Patent Application Publication
(10) Pub. No.: US 2015/0190608 A1
(43) Pub. Date: Jul. 9, 2015

(54) REALTIME FEEDBACK RESPONSE SYSTEM AND METHODS OF USING THE SAME

(71) Applicant: Nurhayat ELTURAN, Berkeley, CA (US)

(72) Inventor: Nurhayat ELTURAN, Berkeley, CA (US)

(21) Appl. No.: 14/578,264

(22) Filed: Dec. 19, 2014

Related U.S. Application Data
(60) Provisional application No. 61/923,383, filed on Jan. 3, 2014.

(51) Int. Cl. A61M 21/02

(52) U.S. Cl. CPC A61M 21/02; A61M 2205/3375; A61M 2205/3303; A61M 2205/3584; A61M 2205/50; A61M 2230/50; A61M 2230/63; A61M 2205/3331; A61M 2205/59; A61M 2209/088; A61M 2230/04; A61M 2230/42; A61M 2205/581; A61M 2205/583

(57) ABSTRACT

Provided herein are apparatus and methods of relaxation. In particular, disclosed herein includes a system that provides realtime or near realtime feedback response to a user in order to achieve a relaxation response. In particular, the realtime feedback response comprises aural signals relating to the user's own heartbeat, breathing, or a combination thereof

### CROSS-REFERENCE TO RELATED APPLICATIONS

This application claims priority from U.S. provisional application No. 61/923,383, filed on Jan. 3, 2014, which is incorporated herein by reference in its entirety.

### FIELD

Provided herein are apparatus and methods of relaxation. In particular, disclosed herein includes a system that provides realtime or near realtime feedback response to a user in order to achieve a relaxation response. In particular, the realtime feedback response comprises aural signals relating to the user's own heartbeat, breathing, or a combination thereof. Further, the realtime feedback response can be combined with visual signals and one or more signals of odor or scent; for example, such signals can each be aimed at enhancing user relaxation. Still further, the realtime feedback response provides a warning when the system is not functioning as intended. The warning can be a sound alert and also include one or more visual signals such as flashing lights or a message.

### BACKGROUND

Mediation can soothe one's mind and alleviate stress. Music, sounds of nature, or one own heart beat can be used to facilitate mediation.

What is needed are portable and wearable systems and methods for soothing a user or facilitating a mediation process by the user.

### SUMMARY

In one aspect, provided herein is a realtime feedback response system for providing relaxation to an individual. For example, the system comprises a data collection component comprising one or more sensors to detect aural signals corresponding to a physiological characteristic of the individual; and a receiving component comprising a listening device.

In some embodiments, the aural signals correspond to a physiological characteristic of an individual. In some embodiments, the receiving component is connected with the delivery component via a wired or wireless connection. In some embodiments, the receiving component receives the aural signals or digital representations thereof from the data collection component, and delivers the aural signals or digital representations thereof to the listening device over an extended period with little or no delay.

In some embodiments, the data collection component further comprises a module for processing the aural signals into digital signals.

In some embodiments, the one or more sensors are selected from the group consisting of a pressure sensor, a position sensor, a force sensor, an angle sensor, a displacement sensor, a distance sensor, a thermal sensor, and a combination thereof.

In some embodiments, the one or more sensors are embedded in a structural element selected from the group consisting of a bracelet, a ring, a chip, a card-like device, an attachment device, and a pendent of a necklace.

In some embodiments, the listening device is selected from the group consisting of a headset, a headphone, one or two ear pieces, and one or two ear buds.

In some embodiments, the data collection component and receiving component communicate via a wireless connection.

In some embodiments, the data collection component and receiving component communicate via a Bluetooth™ connection.

In some embodiments, the physiological characteristic is a cardiac or a breathing pattern. In some embodiments, the aural signals comprise data measured during a cardiac cycle. In some embodiments, the aural signals comprise data measured during a breath-in and breath-out cycle.

In one aspect, provided herein is a method for relaxing an individual by providing realtime feedback response of a physiological characteristic of the individual to the individual. For example, the method comprises the steps of providing a data collection component, and providing a receiving component. In some embodiments, the collection component collects signals or responses from the individual via one or more sensors. In some embodiments, the signals or responses comprise aural signals corresponding to a physiological characteristic of the individual. In some embodiments, the receiving component comprises a listening device and receives the signals or responses or digital representations thereof from the data collection component via wired or wireless communication over an extended period with little or no delay.

In some embodiments, the method further comprises step of collecting the signals or responses via one or more sensors in the data collection component.

In some embodiments, the method further comprises step of transmitting the signals or responses or digital representations thereof, via a wired or wireless communication, from the data collection component to the receiving component.

In some embodiments, the method further comprises step of processing the signals or responses, at the data collection component, into one or more digital representations thereof.

In some embodiments, the data collection component and receiving component are connected by an audio cable.

In some embodiments, the data collection component and receiving component are connected via wireless network.

In one aspect, provided herein is a method of causing an individual to relax by receiving realtime feedback signals or responses of a physiological characteristic of the individual. The method comprises a step of receiving, via a receiving component, signals or responses concerning the physiological characteristic of individual. In some embodiments, the signal or response comprises aural signals corresponding to a physiological characteristic of the individual. In some embodiments, the aural signals are collected in realtime over an extended period. In some embodiments, the aural signals are collected via one or more sensors in a collection component.

In some embodiments, the aural signals are transferred from the collection component to the receiving component. In some embodiments, the receiving component is positioned in close proximity to the collection component.

In some embodiments, the aural signals comprise data measured during a cardiac cycle. In some embodiments, the aural signals comprise data measured during a breath-in and breath-out cycle.

In some embodiments, the data collection component and receiving component communicate via a Bluetooth™ connection.

When applicable, the embodiments described herein can be used in connection with any aspect of the invention.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1A illustrates exemplary embodiments.

FIG. 1B illustrates exemplary embodiments.

FIG. 2 illustrate an exemplary embodiment.

FIG. 3 illustrates an exemplary embodiment.

### DETAILED DESCRIPTION OF THE INVENTION

### Realtime Feedback Response System

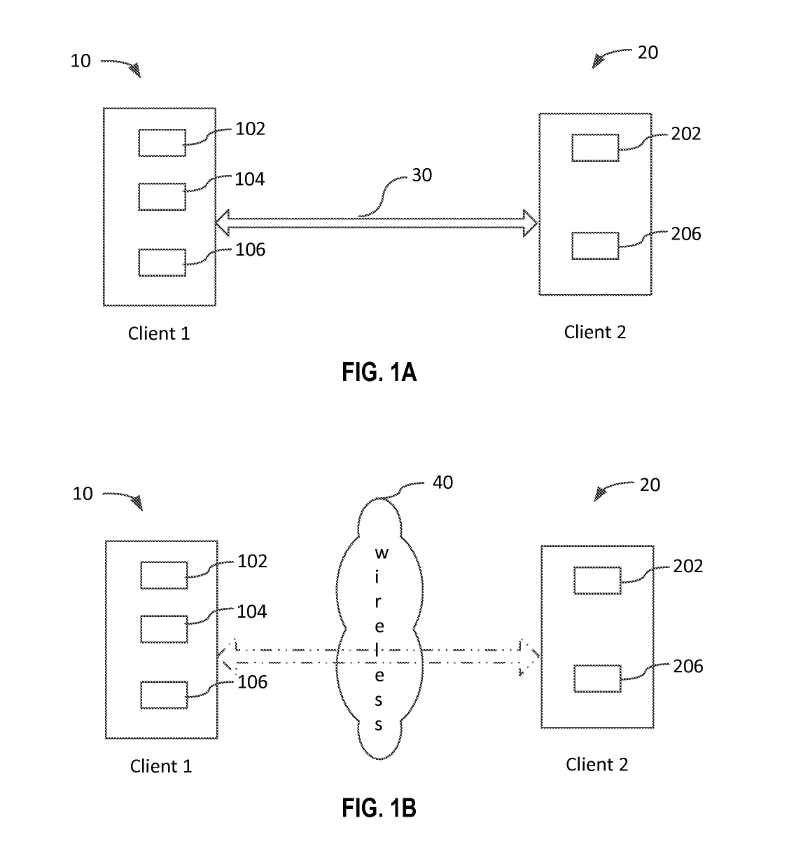

In one aspect, provided herein is a realtime feedback response system. The system comprises a collection component 10 (e.g., element 10 in FIGS. 1A & 1B), a receiving component 20 (e.g., element 20 in FIGS. 1A & 1B). Each component further comprises multiple functional subunits (e.g., 102, 104, 106 in element 10, or 202 and 206 in element 20).

In some embodiments, collection component 10 directly communicates with receiving component 20 by a wired connection. In some embodiments, collection component 10 indirectly communicates with receiving component 20 via a wireless network (e.g., via Bluetooth® connection).

The realtime feedback response system and associated methods are used to provide calm and relaxation to a person in need thereof. For example, the system and methods can be used during mediation, to sooth a person's emotion, and to alleviate insomnia.

As used herein, a user refers to a person who controls the realtime feedback response system. In some embodiments, the user is the individual on whom the realtime feedback response system is applied. In some embodiments, the user is another individual who applies the realtime feedback response system to an individual who is in need of the system. The other individual can be a friend, a family member, or a health care professional.

### Data Collection Component

Referring to FIGS. 1A and 1B, collection component 10 is a functional component by which the realtime feedback response system collect signals to be used for the feedback response system. In some embodiments, such signals comprise aural signals relating to the user's own heartbeat, breathing, or a combination thereof.

According to one aspect of the invention, collection component 10 can take any form, shape and size. For certain applications, collection component 10 is a wearable item that can be placed at the site of collection when signals are collected. For example, the wearable item is a bracelet, a ring, a pin, a brooch, a chip, a card-like device, an attachment device, a pendent of a necklace, and something similar.

In some embodiments, collection component 10 is a bracelet. A sensor module 102 is positioned on the inside of the bracelet. When the realtime feedback response system is in use, a user places sensor module 102 on or near the pulse near the wrist.

In some embodiments, collection component 10 is a pendant on a necklace. A sensor module 102 is positioned on one side of the necklace. When the realtime feedback response system is in use, a user places sensor module 102 on or near the heart. In some embodiments, the sensor patch is placed on the chest of a user when the user is lying down to detect a breathing pattern. In some embodiments, collection component 10 is a ring. In some embodiments, collection component 10 is a patch.

For data measurements, sensor patch 102 includes one or more sensors. For example, the one or more sensor can be an audio sensor, a pressure sensor, a position sensor, a force sensor, an angle sensor, a displacement sensor, a distance sensor, a thermal sensor, and any other applicable sensors. In some embodiments, a combination of one or more sensors of different types are used.

In some embodiments, when the data collection component is used to measure a breathing pattern, a microphone (i.e., an audio sensor) can be used for the measurements. In some embodiments, the microphone is used in combination with another sensor, for example, a position sensor for detecting the rise and fall of the chest when the subject breathes.

In some embodiments, a timer module 104 is included in the collection component. For example, a user can set the timer for a couple of minutes up to hours. A user can set the timer for a period of time (e.g., half an hour) for a calming meditation session during lunch time. A user can also set the timer for a period of time (e.g., 5 minutes or shorter, 10 minutes or shorter, 15 minutes or shorter, 20 minutes or shorter, half an hour or shorter, 45 minutes or shorter, an hour or shorter, 90 minutes or shorter, two hours or shorter, and etc.) before going to bed.

In some embodiments, a monitor module 106 is included in the collection component. For example, once monitor component 106 detects an irregular breathing pattern or cardiac rhythm, an error message is displayed on a display module (e.g., as part of collection component 10). Alternatively or additionally, the monitor component plays a sound when the irregular breathing pattern or cardiac rhythm is detected. A user can reset or re-adjust the collection component to restart data collection.

In some embodiments, a monitor module is used to send an error message to an external device. The external device can be an external monitor, a portable device, a PDA, a beeper, or a cell phone. In some embodiments, the monitor component is linked with a cell phone, so that the error message is sent from the cell phone to the external device. When a trained family member, a nurse, or another health professional receives the message, he or she can reset or re-adjust the monitor component to restart data collection for proper measurement.

In some embodiments, a data processing module is included in the data collection component. The data processing module takes a raw signal and converts into a digital representation of the signal. Exemplary methods for data processing can be found, for example, in U.S. Pat. Pub. No. 2010/0240945 by N. Bikko, which is hereby incorporated by reference in its entirety.

In some embodiments, the data processing module has the capacity to combine signals of different types or digital representations thereof. For example, heartbeats and breathing pattern can be combined and sent to the receiving component. In some embodiments, one or more types of signals or digital representations thereof are combined with an external audio signal, for example, music (the tempo of which is adjusted according to the cardiac rhythms or breathing pattern.

It will be understood by one of skill in the art that any number of different functional modules in the data collection component can be used in any combinations.

### Receiving Component

Referring to FIGS. 1A and 1B, receiving component 20 is a functional component by which the realtime feedback response system receives the signals collected by the data collection component. In some embodiments, such signals comprise aural signals relating to the user's own heartbeat, breathing, or a combination thereof.

In some embodiments, receiving component 20 comprises a listening device, such as an ear phone. In some embodiments, receiving component 20 is directly connected to data collection component 10. In some embodiments, the listening device is selected from the group consisting of a headset, a headphone, one or two ear pieces, one or two ear buds and any applicable device. In some embodiments, a customized listening device with only one ear piece or one ear bud can be used in combination with the data collection component 10. In some embodiments, the listening device is a typical Bluetooth™ mobile phone headset, which is worn on only one ear of the user.

In some embodiments, a regular pair of ear phones, ear bugs or headphones can be used as the listening device; for example, they can be plugged into a standard receiving jacket in the data collection component 10.

In some embodiments, receiving component 20 is connected to data collection component 10 via a wired connection 30; e.g., by an audio cable, a network cable or an electrical cables.

In some embodiments, receiving component 20 is connected to data collection component 10 via a wireless connection 40; e.g., by Bluetooth™ connection, WiFi or infrared signals.

In some embodiments, receiving component 20 receives the information from data collection component 10 in realtime. Here, realtime is defined as having little or no delay between data collection and data received at the listening device. In some embodiments, the delay is less than 10 seconds. In some embodiments, the delay is less than 8 seconds. In some embodiments, the delay is less than 6 seconds. In some embodiments, the delay is less than 4 seconds. In some embodiments, the delay is less than 2 seconds. In some embodiments, the delay is less than 1 second. In some embodiments, the delay is less than 0.8 second. In some embodiments, the delay is less than 0.5 second. In some embodiments, the delay is less than 0.2 second. In some embodiments, the delay is less than 0.1 second. In some embodiments, the delay is less than 0.05 second. In some embodiments, the delay is less than 0.01 second.

In some embodiments, receiving component 20 receives the information from data collection component 10 in a delayed manner. In some embodiments, the delay is 1 cycle or longer (e.g., a breathe-in and breathe-out cycle or a cardiac cycle). In some embodiments, the delay is 2 cycles or longer (e.g., a breathe-in and breathe-out cycle or a cardiac cycle). In some embodiments, the delay is 3 cycles or longer (e.g., a breathe-in and breathe-out cycle or a cardiac cycle). In some embodiments, the delay is 4 cycles or longer (e.g., a breathe-in and breathe-out cycle or a cardiac cycle). In some embodiments, the delay is 5 cycles or longer (e.g., a breathe-in and breathe-out cycle or a cardiac cycle). In some embodiments, the delay is 6 cycles or longer (e.g., a breathe-in and breathe-out cycles or a cardiac cycle). In some embodiments, the delay is 7 cycles or longer (e.g., a breathe-in and breathe-out cycle or a cardiac cycle). In some embodiments, the delay is 8 cycles or longer (e.g., a breathe-in and breathe-out cycle or a cardiac cycle). In some embodiments, the delay is 9 cycles or longer (e.g., a breathe-in and breathe-out cycle or a cardiac cycle). In some embodiments, the delay is 10 cycles or longer (e.g., a breathe-in and breathe-out cycle or a cardiac cycle). In some embodiments, the delay is 12 cycles or longer (e.g., a breathe-in and breathe-out cycle or a cardiac cycle). In some embodiments, the delay is 15 cycles or longer (e.g., a breathe-in and breathe-out cycle or a cardiac cycle). In some embodiments, the delay is 20 cycles or longer (e.g., a breathe-in and breathe-out cycle or a cardiac cycle). In some embodiments, the delay is 25 cycles or longer (e.g., a breathe-in and breathe-out cycle or a cardiac cycle). In some embodiments, the delay is 30 cycles or longer (e.g., a breathe-in and breathe-out cycle or a cardiac cycle).

In some embodiments, when signals of different types or digital representations thereof are received at the receiving component, the listening device played different types of signals simultaneously; for example, sounds corresponding to cardiac rhythms and breathing pattern are played simultaneously. In some embodiments, signals of different types or digital representations thereof are played by the listening device in an alternating fashion. In some embodiments, sounds corresponding to one or more types of signals or digital representations thereof are combined with an external audio signal, for example, music (the tempo of which is adjusted according to the cardiac rhythms or breathing pattern.

In some embodiments, the receiving component has a module for volume control. In some embodiments, the volume control is located on the listening device. In some embodiments, the volume control is located on the connecting cable. In some embodiments, the volume control is located on the data processing component instead of the receiving component.

In some embodiments, the receiving component comprises a timer module. A user can use the time module to decide the duration during which data is communicated to the receiving component. In some embodiments, the timer module is an alternative to the timer module on the data collection component. In some embodiments, the timer module is used in additional to the timer module on the data collection component. For example, the timer module on the receiving component can overwrites a pre-set time period set by the timer on the data collection component.

### Methods

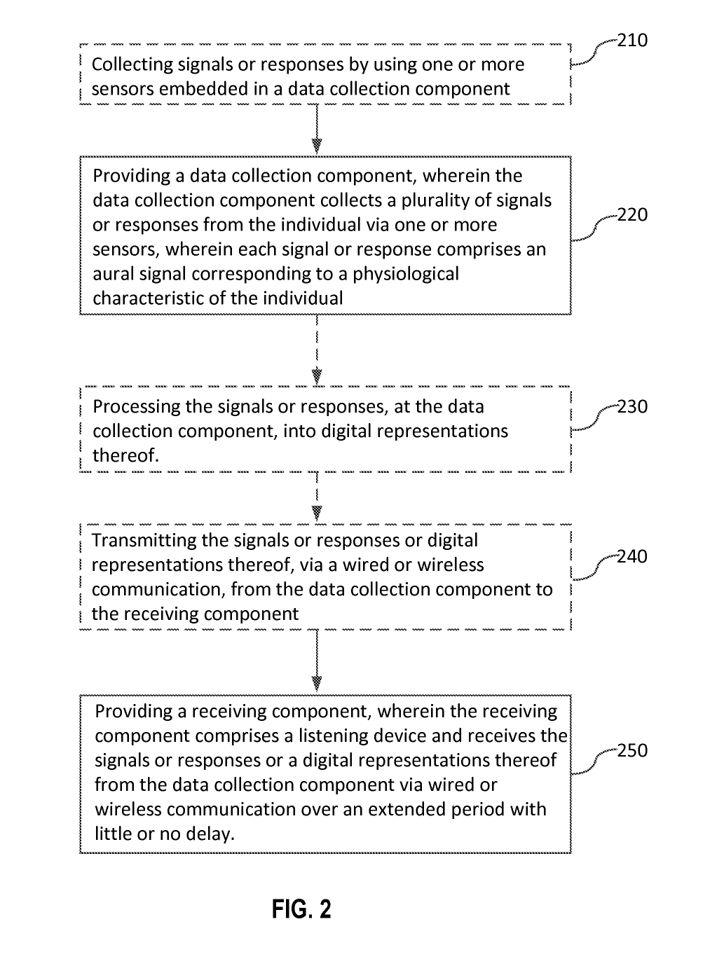

FIG. 2 illustrate an exemplary process for practicing the current invention. At sometimes optional step 210, a data collection component is used to collection signals or responses. The data collection component comprises one or more types of sensors. Exemplary sensor types include but are not limited to a microphone or another aural sensor, a pressure sensor, a position sensor, a force sensor, an angle sensor, a displacement sensor, a distance sensor, a thermal sensor, or a combination thereof.

At step 220, the data collection component collects a plurality of signals or responses from an individual. Each of the signal or response comprises an aural signal corresponding to a physiological characteristic of the individual. For example, the physiological characteristic can be a cardiac or a breathing pattern. In some embodiments, the cardiac patterns comprises multiple cycles of cardiac cycles, each including a complete heartbeat from its generation to the beginning of the next heartbeat. In some embodiments, the corresponding characteristic is the number of cardiac cycles within a predetermined period of time. The predetermined period of time can be 15 seconds, 30 seconds, 45 seconds, a minute, 2 minutes, 5 minutes, or any duration of time in which the number of cardiac cycles are counted. In some embodiments, the corresponding characteristic is the time between a first heartbeat and the second heartbeat immediately following the first heartbeat.

In some embodiments, the corresponding characteristic is the number of breaths taken within a predetermined period of time. The predetermined period of time can be 15 seconds, 30 seconds, 45 seconds, a minute, 2 minutes, 5 minutes, or any duration of time in which the number of cardiac cycles are counted. In some embodiments, the corresponding characteristic is the inhaling time within a single breath cycle. In some embodiments, the corresponding characteristic is the exhaling time within a single breath cycle.

At sometimes optional step 230, data collected by the sensors are subject to processing by a data processing module; e.g., within the data collection component. At this step, raw data collection are converted into corresponding digital representations. For example, raw breathing sounds collected by a microphone can be converted into digital signals that correspond to the inhaling and exhaling pattern. In some embodiment, the data processing module is independent from but connected with the one or more sensors for data collection. In some embodiments, the data processing module and one or more data collection sensors are combined; for example, in one integrated chip.

At sometimes optional step 240, raw data collected by the sensor or digital representations of the raw data are transmitted from the data collection component to the receiving component, via wired or wireless communication. In some embodiments, the wired communication is achieved by an audio cable connecting the data collection component and receiving component.

At step 250, the raw data collected by the sensor or digital representations of the raw data are received at a receiving component. In some embodiments, the receiving component comprises multiple functional modules, including but not limited to a listening device. In its simplest form, the listening device can be a headset, one or two earphones or one or more ear buds. In some embodiments, the listening device is a typical Bluetooth™ enabled ear piece.

In some embodiments, data are communicated to the receiving component over an extended period of time. The extended period of time can be set before a user start a treatment session. For example, the treatment session can be 5 minutes or shorter, 10 minutes or shorter, 15 minutes or shorter, 20 minutes or shorter, half an hour or shorter, 45 minutes or shorter, an hour or shorter, 90 minutes or shorter, two hours or shorter and etc.

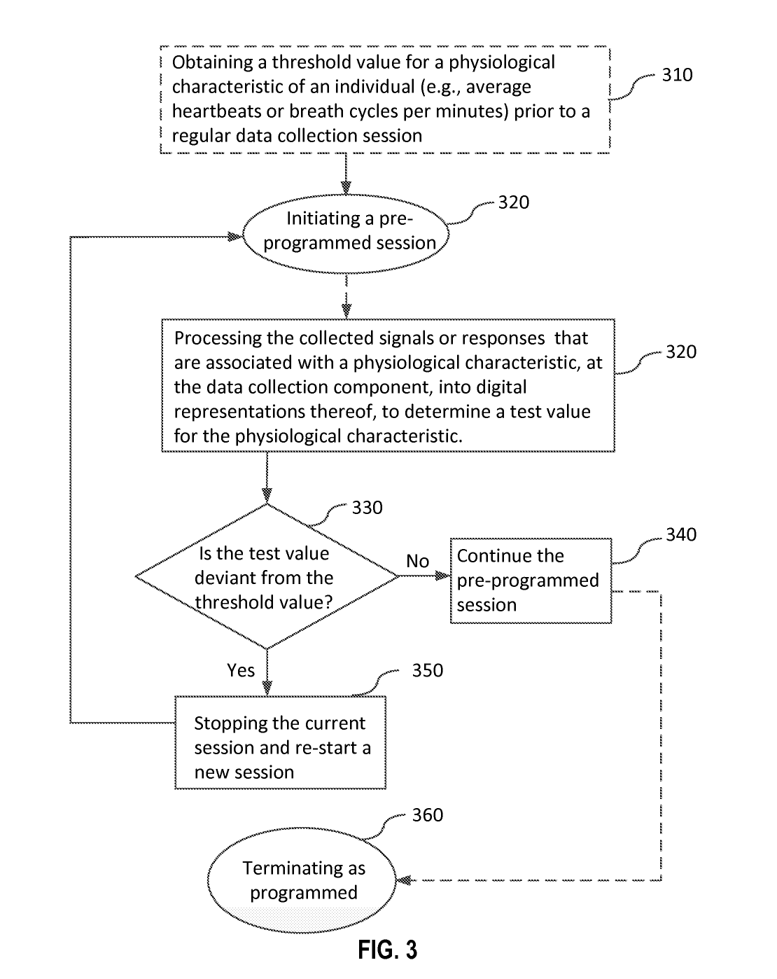

In some embodiments, an optional calibration mechanism is implemented to ensure proper working of the system or to detect any irregularities. FIG. 3 illustrates an exemplary calibration process. In some embodiments, the calibration mechanism is used to detect malfunctioning of the system. In some embodiments, the calibration mechanism is used to detect irregularities in the individual being treated.

At sometimes optional step 310, a pre-treatment session is applied before a regular data collection session of an actual pre-programmed treatment session. The pre-treatment session can be shorter than the actual pre-programmed treatment session; for example, only 2 or 5 minutes. During the pre-treatment session, a threshold value for a physiological characteristic an individual is obtained. In some embodiments, the threshold is the average heartbeats per minute. In some embodiments, the threshold is the average number of breath cycles per minute. In some embodiments, the threshold values are measured multiple times.

At step 320, a pre-programmed treatment session is initiated. At step 330, data collected corresponding to a physiological characteristic are processed to determine a test value. In some embodiments, the test value is the average heartbeats per minute. In some embodiments, the test value is the average number of breath cycles per minute. In some embodiments, the threshold values are measured multiple times.

At step 340, the test value from step 330 is compared with the pre-determined threshold value from step 310 to determine whether the test value is a deviant of the threshold value. In some embodiments, the test value is deviant if it is more than 15% higher or lower than the threshold value. In some embodiments, the test value is deviant if it is more than 20% higher or lower than the threshold value. In some embodiments, the test value is deviant if it is more than 25% higher or lower than the threshold value. In some embodiments, the test value is deviant if it is more than 30% higher or lower than the threshold value. In some embodiments, the test value is deviant if it is more than 40% higher or lower than the threshold value. In some embodiments, the test value is deviant if it is more than 50% higher or lower than the threshold value. In some embodiments, the test value is deviant if it is more than 60% higher or lower than the threshold value. In some embodiments, the test value is deviant if it is more than 70% higher or lower than the threshold value. In some embodiments, the test value is deviant if it is more than 80% higher or lower than the threshold value. In some embodiments, the test value is deviant if it is more than 90% higher or lower than the threshold value. In some embodiments, the test value is deviant if it is more than 100% higher or lower than the threshold value.

If the test value is not deviant from the threshold value, the pre-programmed treatment session continues until it naturally stops at step 370.

If the test value is deviant from the threshold value, the pre-programmed treatment session is stopped at step 360 and the pre-programmed treatment session is re-initiated from step 320.

In some embodiments, when the test value is significantly deviant from the threshold value, an alarm message will be sent to the user, a healthcare professional or anyone granted access for such information.

### Applications

The system and methods disclosed herein can be used to provide calm and relaxation to an individual in need. For example, an user can wear the device during a meditation session or just before going to bed. In some embodiments, the methods and system can be applied by a healthcare profession to an agitated patient; for example, patient with dementia, or attention deficient syndrome.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

We claim:

1. A realtime feedback response system for providing relaxation to an individual, comprising:
a data collection component comprising one or more sensors to detect aural signals corresponding to a physiological characteristic of the individual; and
a receiving component comprising a listening device;
wherein the aural signals correspond to a physiological characteristic of an individual,
wherein the receiving component is connected with the delivery component via a wired or wireless connection, and
wherein the receiving component receives the aural signals or digital representations thereof from the data collection component, and delivers the aural signals or digital representations thereof to the listening device over an extended period with little or no delay.

2. The realtime feedback response system of claim 1, wherein the data collection component further comprises a module for processing the aural signals into digital signals.

3. The realtime feedback response system of claim 1, wherein the one or more sensors are selected from the group consisting of a pressure sensor, a position sensor, a force sensor, an angle sensor, a displacement sensor, a distance sensor, a thermal sensor, and a combination thereof.

4. The realtime feedback response system of claim 2, wherein the one or more sensors are embedded in a structural element selected from the group consisting of a bracelet, a ring, a chip, a card-like device, an attachment device, and a pendent of a necklace.

5. The realtime feedback response system of claim 4, wherein the listening device is selected from the group consisting of a headset, a headphone, one or two ear pieces, and one or two ear buds.

6. The realtime feedback response system of claim 1, wherein the data collection component and receiving component communicate via a wireless connection.

7. The realtime feedback response system of claim 6, wherein the data collection component and receiving component communicate via a Bluetooth™ connection.

8. The realtime feedback response system of claim 1, wherein the physiological characteristic is a cardiac or a breathing pattern.

9. The realtime feedback response system of claim 1, wherein the aural signals comprise data measured during a cardiac cycle.

10. The realtime feedback response system of claim 1, wherein the aural signals comprise data measured during a breath-in and breath-out cycle.

11. A method for relaxing an individual by providing realtime feedback response of a physiological characteristic of the individual to the individual, comprising:
providing a data collection component, wherein the collection component collects signals or responses from the individual via one or more sensors, wherein the signals or responses comprise aural signals corresponding to a physiological characteristic of the individual; and
providing a receiving component, wherein the receiving component comprises a listening device and receives the signals or responses or digital representations thereof from the data collection component via wired or wireless communication over an extended period with little or no delay.

12. The method for relaxing an individual of claim 11, further comprising:
collecting the signals or responses via one or more sensors in the data collection component.

13. The method for relaxing an individual of claim 11, further comprising:
transmitting the signals or responses or digital representations thereof, via a wired or wireless communication, from the data collection component to the receiving component.

14. The method for relaxing an individual of claim 11,
processing the signals or responses, at the data collection component, into one or more digital representations thereof.

15. The method for relaxing an individual of claim 11, wherein the data collection component and receiving component are connected by an audio cable.

16. The method for relaxing an individual of claim 11, wherein the data collection component and receiving component are connected via wireless network.

17. A method of causing an individual to relax by receiving realtime feedback signals or responses of a physiological characteristic of the individual, comprising:
receiving, via a receiving component, signals or responses concerning the individual;
wherein the signal or response comprises aural signals corresponding to a physiological characteristic of the individual,
wherein the aural signals are collected in realtime over an extended period,
wherein the aural signals are collected via one or more sensors in a collection component,
wherein the aural signals are transferred from the collection component to the receiving component;
and wherein the receiving component is positioned in close proximity to the collection component.

18. The method of claim 17, wherein the aural signals comprise data measured during a cardiac cycle.

19. The method of claim 17, wherein the aural signals comprise data measured during a breath-in and breath-out cycle.

20. The method of claim 17, wherein the data collection component and receiving component communicate via a Bluetooth™ connection.
